(19) United States
(12) Patent Application Publication
(10) Pub. No.: US 2009/0220949 A1
(43) Pub. Date: Sep. 3, 2009

(54) Mutations Associated with the Long QT Syndrome and Diagnostic Use Thereof

(75) Inventor: Silvia Giuliana Priori, Milano (IT)

Correspondence Address: CLARK & ELBING LLP, 101 FEDERAL STREET, BOSTON, MA, 02110, US

(73) Assignees: Fondzione Salvatore Maugeri Clinica Del Lavoro E Della Riabilitazione I.R.C.C.S., Pavia (IT); Universita' Degli Studi Di Pavia, Pavia (IT)

(21) Appl. No.: 11/916,791

(22) PCT Filed: Jun. 7, 2006
(86) PCT No.: PCT/EP2006/062956

§ 371 Date: Dec. 4, 2008

(30) Foreign Application Priority Data
Jun. 7, 2005 (IT) MI2005A001047

(51) Int. Cl. C12Q 1/68; B01D 57/02

(52) U.S. Cl. 435/6; 204/456; 536/23.1

(57) ABSTRACT

The present invention is based on the identification of new mutations in KCNQ1 (also termed KvLQTI), KCNH2 (also termed HERG), SCN5A, KCNE1 (also termed minK), KCNE2 (also termed MiRP) genes that encode ionic channels involved in cardiac electrical activity and are potentially responsible for the Long QT Syndrome. According to a main aspect, the invention relates to nucleic acids, oligonucleotides and polynucleotides and mRNA, containing sequences of KCNQ1, KCNH2 SCN5A, KCNE1, KCNE2 genes and cDNAs in a mutated form and to respective variant proteins thereof. A preferred embodiment of the present invention is represented by a diagnostic method based on the identification of a group of about 70 non-private mutations in the KCNQ1, KCNH2 and SCN5A genes, detected at high frequency. The method, which is able to identify about 40% of the probands, is non exclusively based on identification of mutations that are described and characterized in this invention where said identification has both prognostic and diagnostic value for the Long QT Syndrome.

### FIELD OF THE INVENTION

The invention relates to new genetic mutations in KCNQ1, KCNH2, SCN5A, KCNE1, KCNE2 genes and to diagnostic tests for their identification.

### STATE OF THE ART

The Long QT Syndrome is an inherited disease predisposing to cardiac arrhythmias and sudden death at a young age. It is characterized by a prolonged QT interval on the electrocardiogram.

Two phenotypic variants have been recognized: an autosomal dominant variant known as Romano Ward Syndrome (RWS) and an autosomal recessive variant termed Jervell Lange Nielsen Syndrome (JLNS) (Romano C et al. Clin Ped 1963; 45:656-657; Ward D C. J Irish Med As 1964; 54:103; Jervell A & Lange-Nielsen F. Am. Heart J 1957; 54:59-61). More recently, two other forms with extra-cardiac involvement have been reported (Splawski I et al. Cell 2004; 119:19-31; Plaster N M et al. Cell 2001; 105:511-519).

The genetic loci associated with the first type of pathology have been located on chromosomes 3, 4, 7, 11 and 21 and the respective genes have been identified. The gene associated with the LQT 1 locus is KCNQ1 (formerly termed KvLQT1), the gene associated with the LQT2 locus is KCNH2 (formerly termed HERG), the gene associated with the LQT3 locus is SCN5A, the gene associated with the LQT4 locus is ANK2, the gene associated with the LQT5 locus is KCNE1 (formerly termed minK) and finally the gene associated with the LQT6 locus is KCNE2 (formerly termed MIRP). All the genes involved encode ion channels (KCNQ1, KCNH2, SCN5A, KCNE1, KCNE2) except for the gene encoding the cardiac form of “ankyrin”, a structural protein which anchors ion channels to the cell membrane (ANK2): however there are very few patients (4-5 families world-wide) showing an involvement of this protein, therefore it is not possible to perform genotype—phenotype relation studies in such a small population.

So far, several mutations in the genes encoding the five ion channels have been reported: for instance US2005/003445 describes mutations in KCNQ1 (or KvLQT1), KCNE1 (or Min K), KCNE2 (or MIRP), KCNH2 (or HERG) and SCN5A genes.

Currently, the diagnosis of Long QT Syndrome is primarily based on identification in a surface electrocardiogram of a heart-rate corrected QT prolongation (QTc≧440 msec for males and QTc≧460 for females). Prolongation of the QT interval may or may not be associated with symptoms linked to the presence of arrhythmias, such as syncopal episodes, however the finding of a prolonged QT interval remains the basic diagnostic element in the disease. However, epidemiological data have shown that only 70% of the subjects affected by this Syndrome displays a prolonged QT interval, therefore it can be deduced that genetic diagnosis is a fundamental tool for the diagnosis of disease at the pre-symptomatic stage. Moreover, since the type of underlying genetic defect affects the seriousness of the Long QT Syndrome and the response to the therapy, it is also evident the importance of molecular diagnosis for assessment of the arrhythmic risk and the following therapeutic choice.

However, a limit to the diffusion of molecular diagnosis is represented by the high number of mutations (for a list see http://pc4.fsm.it:81/cardmoc/) that can cause the disease, many of which are “private” mutations found in a single patient or family. So far, this has made necessary to screen the entire coding region (ORF) of all genes involved in the Syndrome. Such an approach is expensive and requires a very log time to formulate the report.

### SUMMARY OF THE INVENTION

The present invention relates to novel nucleotide mutations in KCNQ1, KCNH2, SCN5, KCNE1, KCNE2 genes that are associated with the full-blown Long QT Syndrome or are associated with the predisposition to said syndrome or with the susceptibility to develop arrhythmias during exposure to trigger-events (food, drugs), and to a method to identify such mutations. Said mutations affect the coding region of the above defined genes and always result in corresponding amino acid changes, leading to the expression of variant proteins with altered functionality compared to wild type proteins.

According to a further primary aspect, the invention relates to a method for identification of about 40% of the carriers of the Long QT Syndrome or of carriers of a predisposition to said Syndrome. Such method involves the detection of a group of about 70 non-private mutations. According to a further aspect, the invention relates to a method for identification of about 20% of the carriers of the Long QT Syndrome or of carriers of a predisposition to said syndrome, comprising the detection of a further selection of about 20 non-private mutations. The detection is performed by well known techniques for identification of point mutations, insertions, deletions, duplications.

Nucleic acids containing previously unreported mutations, vectors containing said nucleic acids and cells transformed with said vectors are also included in the invention.

According to a further aspect, the invention relates to the detection, at the amino acid sequence level, of novel mutations or of different groups of non-private mutations.

### DESCRIPTION OF THE FIGURES

FIG. 1 shows the distribution of QT intervals in subjects not affected by LQTS and, in red, in subjects affected by LQTS. A wide overlap of the QT interval duration curve between affected and non affected subjects is noticed, therefore only genetic analysis can allow a correct diagnosis in affected subjects with a QT within normal limits.

FIG. 2. Nucleotide and amino acid sequences of wild type KCNQ1 cDNA.

FIG. 3. Nucleotide and amino acid sequences of wild type KCNH2 cDNA.

FIG. 4. Nucleotide and amino acid sequences of wild type SCN5 cDNA.

FIG. 5. Nucleotide and amino acid sequences of wild type KCNE1 cDNA.

FIG. 6. Nucleotide and amino acid sequences of wild type KCNE2 cDNA.

### DETAILED DESCRIPTION OF THE INVENTION

For the purpose of the present invention, the following definitions have been used:

Mutation: for the purpose of the present invention, it is meant by mutation, unless otherwise indicated, any change of the nucleotide sequence, involving one or more nucleotides, hence including a permutation, insertion or deletion that is absent in the DNA from control individuals or in wild type DNA corresponding to the cDNA sequences deposited in the GenBank with accession numbers: AF00571 (cDNA KvLQT1; gene: KCNQ1: AJ006345), NM005136 (cDNA: MiRP; gene: KCNE2, AB009071), NM000335 (cDNA: Nav1.5; gene: SCN5A NT_{—}022517.17), NM000238 (cDNA: HERG; gene: KCNH2: NT_{—}011512.10), NM00219 (KCNE1; gene: AP000324), resulting also in an amino acid sequence change in the encoded protein.

In the present invention, unless otherwise indicated, the positions of a single mutated nucleotide or of several mutated nucleotides are indicated with the number of the mutated nucleotide (like, for instance, in table 1 where the reference of the identifying sequence can be found, with the mutated nucleotide underlined) or with the respective codon or with the amino acid affected by the mutation: therefore, the name of the P345 mutation refers to the amino acid change, which is proline in wild type, as well as to the mutation of one of the nucleotides of the proline codon at position 345 of the amino acid sequence: in this case, the specific mutation of the nucleotide sequence is also reported.

In the present invention, the definitions “locus” and “gene” are used interchangeably and they correspond to, respectively: LQT1 locus—KCNQ1 (o KvLQT1) gene, LQT2 locus—KCNH2 (o HERG) gene, LQT3 locus—SCN5A gene, LQT5 locus—KCNE1 (o minK) gene, LQT6 locus—KCNE2 (o MiRP) gene.

QTc by QTc it is meant the QT interval corrected for the heart rate expressed as RR interval according to the formula QTc=QT/v RR.
IQR: Interquartile range. Values corresponding to the 75th percentile and the 25th percentile of a variable are reported under this definition.
Long QT Syndrome (QTS) Two phenotypic variants of the Long QT Syndrome have been recognized: an autosomal dominant variant known as Romano Ward Syndrome (RWS) and an autosomal recessive variant termed Jervell Lange Nielsen Syndrome (JLNS). In addition, two other forms with extra-cardiac involvement have been reported.
ORF: Open Reading Frame=the coding portion of a gene.

The present invention is based on the identification of new mutations in KCNQ1 (also termed KvLQT1), KCNH2 (also termed HERG), SCN5A, KCNE1 (also termed minK), KCNE2 (also termed MiRP) genes encoding ion channels involved in the control of cardiac electrical activity and particularly in generation of the cardiac action potential. A genetically based dysfunction of the proteins encoded by these genes can cause the Long QT Syndrome.

Therefore, according to a first aspect, the invention relates to 139 new mutations of the coding region in the genomic DNA corresponding to KCNQ1, KCNH2 SCN5A, KCNE1, KCNE2 genes, enlisted in table 1, to nucleic acids, either RNA or DNA, and preferably cDNA and genomic DNA comprising the specific mutations and encoding the entire protein in variant forms with altered function compared to the wild type protein and with the potential to cause the Long QT Syndrome. Moreover, the invention relates to nucleic acid fragments and their encoded proteins, characterized in that they comprise at least one of the amino acid changes reported in Table 1 (SEQ ID NO: 11-149) and are useful for diagnostic or research purposes.

The invention also refers to fragments, polynucleotides and oligonucleotides, comprising at least the 9-nucleotide sequence reported in Table 1 (SEQ ID NO: 11-149) including the mutation and, alternatively or optionally, depending on their use, the adjacent nucleotides that can be derived from the sequences of KCNQ1, KCNH2 SCN5A, KCNE1, KCNE2 genes or cDNAs as described for wild type.

In a preferred embodiment, the length of the oligonucleotides of the invention, which are preferably used for diagnostic purposes, is shorter than or equal to 50 nucleotides, preferably between 40 and 15 nucleotides, even more preferably between 30 and 20 nucleotides. These oligonucleotides comprise the nonanucleotides defined in Table 1 or oligonucleotides suitable for detection of position, structure and type of mutations defined therein. Suitable oligonucleotides can be designed by an expert in the field based on the mutated sequences of in the present invention, on the published sequence of each wild type gene, whose accession number is herein reported, and on his/her own knowledge of the field. The oligonucleotides of the invention, and/or their complementary sequences, are chemically synthesized and can comprise chemically modified nucleotides (for instance phosphorothioated nucleotides) or a fluorochrome or chromophore label, preferably at the 5′ and/or 3′ terminus.

Such oligos can be used for gene amplification reactions or for hybridization in homogeneous or heterogeneous phase: they can be used as such or in a form bound to a solid matrix or a two-dimensional or three-dimensional support, for instance a membrane, or to the bottom of a well in a plate or to a microchip.

The nucleic acids of the invention are double or single stranded: wherein single stranded molecules include also oligonucleotides and complementary DNA (cDNA) or antisense DNA.

The nucleic acids of the invention, particularly the cDNA and its fragments, comprising at least one mutation according to the invention, can be cloned into vectors, for instance expression vectors for the production of high amounts of recombinant protein useful for functional characterization of different variants or to set up immunoassays.

Therefore, vectors containing the nucleotide sequences and cells transformed with such vectors, and expressing the mutant proteins, are also comprised in the invention.

The nucleic acids and proteins of the invention are claimed for diagnostic use in the Long QT Syndrome, particularly in the Romano Ward Syndrome and/or the Jervell Lange-Nielsen Syndrome in in vitro methods. Moreover, the recombinant proteins and their fragments comprising the mutation are useful for production of specific antibodies against the mutant protein, which are able to specifically bind the mutated but not the wild type protein. Together with the proteins, said antibodies are used to set up diagnostic immunoassays in vitro.

Therefore, according to a preferred embodiment, the invention relates to a method for identification in a sample of at least 1 of the mutations in Table 1, where such identification has both prognostic and diagnostic value for the Long QT Syndrome, in particular for the Romano Ward and/or Jervell Lange types for example by hybridization or by PCR. The identification of at least one of the mutations reported in Table 1 is carried out according to molecular methods well known in the art. The presence of said mutations in the nucleic acids of the sample correlates with a predisposition to develop such disease or with the full-blown disease.

The sample is preferably represented by nucleic acids purified from a biological sample, such as cells obtained from biological fluids, as for instance blood or other tissues. The nucleic acids are preferably genomic DNA or mRNA. In the latter case, the sample can be retrotranscribed into cDNA prior to sequence analysis.

A further aspect of the invention relates to a diagnostic method based on the identification of a group of about 70 non-private mutations in KCNQ1, KCNH2 and SCN5A genes, selected among new mutations shown in Table 1, and mutations well known in the art, selected among those detected by the authors of the present invention, which occur at high rate in a statistically significant sample of probands and which are able to identify about 40% of the probands.

According to a further aspect, the method to diagnose the Long QT Syndrome of RW and/or Jervell Lange type, or the genetic predisposition to said syndrome which represents the genetic cause of QT interval alterations found in an electrocardiogram, comprises at least the identification of hot spot mutations as defined below. Hot spot mutations are the most commonly found according to the population studied and occur in at least 3 or more clinically affected individuals of different families. In the KCNQ1 gene, such hot spot mutations affect the codons encoding for: R190, preferably R190W, where even more preferably W is encoded by the corresponding codon in sequence SEQ ID NO: 17, R231C and more preferably R231H, where even more preferably H is encoded by the corresponding codon in sequence SEQ ID NO: 24, V254 more preferably V254M and V254L, where even more preferably L is encoded by the corresponding codon in sequence SEQ ID NO: 26, and so on according to the preferred embodiments enlisted in Table 2 for the following mutations in the KCNQ1 gene: G269, S277, G314, A341, A344; in the KCNH2 gene in codons: A561, G572 (preferably identified by sequence SEQ ID NO: 97), G628; in the SCN5A gene in codons P1332 and E1784.

Hot spot mutations characterize 24% of the probands with electrocardiographic alterations; therefore the present invention comprises a method for identification of hot spot mutations as defined in the present invention which make use of methods well known in the art. In the KCNQ1 gene, the rate of said hot spot mutations in the sample is the following for each indicated codon: 190 (n=12), 231 (n=4) 254 (n=4), 269 (n=4), 277 (n=5), 314 (n=4), 341 (n=6), 344 (n=9), in the KCNH2 gene it is for codon 561 (n=7), 572 (n=4) and 628 (n=7); in the SCN5A gene is the following for each indicated codon: 1332 (n=5) and 1784 (n=3).

It should be noted that mutations in KCNQ1 and KCNH2 (LQT1 and LQT2) genes are more common in patients with Long QT Syndrome; they account for the genetic cause in 90% of these pathologies.

The preferential search identification of mutations in one of the hot spot codons, or of the mutations listed in Table 2, allows a rapid and highly cost-effective diagnosis of the Long QT Syndrome, with remarkable reduction of costs and expansion of the diagnostic potential to the general population.

In a particularly preferred aspect, the invention relates to a method for the molecular diagnosis (carried out on the nucleic acids of the patient) of the Long QT Syndrome, particularly the inherited forms Romano-Ward and/or Jervell Lange, comprising the identification of a group of non-private mutations (i.e. found in at least two individuals belonging to different families) affecting the following codons or groups of codons:
in the KCNQ1 gene: L137 (exon 2), R174, G179, R190 (exon 3), I204 (exon 4), R231, D242, V254, H258, R259 (exon 5), L262, G269, S277, V280, A300, W305, (exon 6), G314, Y315, T322, G325, A341, P343, A344 (exon 7), R360 (exon 8), R518 (exon 12), R539 (exon 13), I567 (exon 14), R591, R594 (exon 15), according to the numbering of codons or amino acids, and according to the numbering of nucleotides with mutation 1514 +1G>A, identified by oligonucleotide SEQ ID NO: 52, 1513-1514delCA, identified by oligonucleotide SEQ ID NO: 53, with mutation 921+1 G>A and with mutation 921+2 T>C;
in the KCNH2 gene: Y43, E58, IAQ82-84 (exon 2), W412, S428 (exon 6), R534, L552, A561, G572, R582, G604, D609, T613, A614, T623, G628 (exon 7), S660 (exon 8), R752 (exon 9), S818, R823 (exon 10) according to the numbering of codons or amino acids, and according to the numbering of nucleotides with mutation 453delC, 453-454insCC, 576delG identified by the oligonucleotide with sequence SEQ ID NO: 79, 578-582deICCGTG identified by the oligonucleotide with sequence SEQ ID NO: 80, G2398+3A>G identified by the oligonucleotide with sequence SEQ ID NO: 110, G2398+3A>T identified by the oligonucleotide with sequence SEQ ID NO: 111, 3093-3106del identified by the oligonucleotide with sequence SEQ ID NO: 125, 3093-3099del/insTTCGC identified by the oligonucleotide with sequence SEQ ID NO: 126, and 3100delC identified by the oligonucleotide with sequence SEQ ID NO: 128;
In the SCN5A gene: A413 (exon 10), T1304, P1332 (exon 21), 1505-1507del (exon 26), R1623 (exon 10), R1644, Y1767, E1784 (exon 28),
   where the presence of a mutation in at least one of the above-mentioned codons or positions indicates the presence of a functional abnormality of the ion channel and that the subject carrying such mutation is affected by or predisposed to the Long QT Syndrome, preferably of the Romano-Ward and/or Jervell Lange type. Table 2 outlines the group of most representative mutations used for the diagnostic methods described above.

In a further embodiment the invention also relates to a two-dimensional or three-dimensional support comprising oligonucleotides capable of selectively detecting the mutations defined in Table 2. According to an even more preferred embodiment, said support comprises polynucleotides or oligonucleotides comprising at least one of the preferred nonanucleotides chosen from those that are most commonly found in the sequences of the probands and reported in Table 1: SEQ ID NO: 13, SEQ ID NO: 16, SEQ ID NO: 17, SEQ ID NO: 21, SEQ ID NO: 24, SEQ ID NO: 26, SEQ ID NO: 27, SEQ ID NO: 28, SEQ ID NO: 29, SEQ ID NO: 34, SEQ ID NO: 39, SEQ ID NO: 40, SEQ ID NO: 41, SEQ ID NO: 46, SEQ ID NO: 55, SEQ ID NO: 56, SEQ ID NO: 59, SEQ ID NO: 67, SEQ ID NO: 69, SEQ ID NO: 70, SEQ ID NO: 71, SEQ ID NO: 75, SEQ ID NO: 79, SEQ ID NO: 80, SEQ ID NO: 85, SEQ ID NO: 88, SEQ ID NO: 97, SEQ ID NO: 98, SEQ ID NO: 99, SEQ ID NO: 106, SEQ ID NO: 110, SEQ ID NO: 111, SEQ ID NO: 113, SEQ ID NO: 125, SEQ ID NO: 128, SEQ ID NO: 133, SEQ ID NO: 134, SEQ ID NO: 140, SEQ ID NO: 142, or their complementary sequences. Alternatively, an expert in the field can design the sequence of oligonucleotides capable of detecting the mutations defined in the present invention, for example by software tools.

According to a further embodiment, the invention comprises a support wherein polynucleotides and/or oligonucleotides include at least one of the nonanucleotides with sequence from SEQ ID NO: 11 to SEQ ID NO: 149 or at least one of their complementary oligonucleotides.

Table 2 shows both the position of the mutated nucleotide and the position of the codon which, as result of the mutation, is different from the wild type. In Tables 1 and 2 it is also possible to identify, in patients affected by the Long QT Syndrome, one or more amino acids preferably found as result of mutations in the codons of a gene.

According to a further aspect the invention comprises the use of a oligonucleotide comprising anyone of the nonamer with wild type sequence corresponding to those identified in SEQ ID NO: 11-149 and in Table 2 for the diagnosis of a full-blown Long QT Syndrome or for the diagnosis of a genetic predisposition to the Long QT Syndrome in in vitro methods.

For the purpose of the present invention it is intended to be comprised within the disclosure of the present invention any nucleotidic mutation leading to any amino acid change different from wild type, in the same position, as herein disclosed, with the exclusion of the mutations already well-known (see references of Table 2) regardless of the codons that are preferably generated as the result of the specific mutation.

Thus, just as an example, the present invention comprises all the mutations affecting, in the case of the KCNQ1 gene, the histidine codon at position 258 (wild type) which, according to the invention, changes from a histidine codon to a different codon that, according to a preferred embodiment, is an arginine (Arg or R) codon, if the mutation affects the second nucleotide of the CAC codon (His), thus changing from A into G (A→G) and producing a CGC codon which encodes for Arg; in the same starting codon a different change, C→A produces a AAC codon which enclodes for Asp as result of a mutation of the first nucleotide of the codon. In this case the mutation according to the invention identifies any amino acid at position 258 that is different from histidine, and preferably identifies arginine or asparagine. Table 2 shows the preferred embodiments for each mutation.

All nucleotide mutations (generally in the third-nucleotide of the codon) which, due to the degeneracy of the genetic code, change the codon giving rise to the same amino acid, identical to the amino acid preferred in the protein sequence, are also intended to be comprised in the present invention. Just as an example, in the case of the mutation of codon 258 in the KCNQ1 gene, already used in the previous example, all the permutations, due to genetic code degeneracy, that change the CAC codon into any of the codons encoding Arginine, or, in the second case, into any of the codons encoding Asp, are intended to be comprised in the invention. Each preferred embodiment of the mutations identified as being related to the Long QT Syndrome found and used in the method of the invention enlisted in Table 1 which reports the mutated nucleotide and/or amino acid according to the nucleotide or amino acid numbering of the corresponding wild type gene sequence which is reported as annex in the Sequence List from SEQ ID NO: 1 to 10.

The identification, according to the invention, of the most representative mutations in subjects at risk for the Long QT Syndrome can be carried out also on the protein product, corresponding to the various amino acid variants, using methods well known in the art, as for instance specific antibodies or differences in the electrophoretic migration pattern.

Based on the findings of the authors of the present invention, which have been also confirmed in an independent sample, at least 40% of mutation carriers (or probands) carries a mutation of one of the codons or one of the mutations reported in Table 2. Therefore, this molecular method represents the first level of molecular screening for the Long QT Syndrome.

The molecular method according to the invention involves also a second level of investigation carried out preferentially on subjects that are found to be negative at the first level screening. Such second level of investigation consists in the characterization of sequences of the Open Reading Frames in KCNQ1 and KCNH2 genes. Finally, the molecular method according to the invention involves a third level for subjects that turned out to be negative in the second level, comprising the analysis of the genes responsible for the less prevalent genetic variants of LQTS, that is for SCN5A, KCNE1 and KCNE2 genes. Said third level can include a confirmation of the sequences of SCN5A, KCNE1 and KCNE2 gene ORFs, for instance by direct sequencing following gene amplification with primer oligonucleotides which can be derived, by methods well known in art, from the published sequence. According to a preferred embodiment, the primers used for direct sequencing of exons are listed in Table 4.

The identification performed with molecular methods according to the present invention can be associated with other measurements or other clinical diagnostic/prognostic methods.

In this respect, the authors of the present invention have evaluated in parallel the sensitivity and specificity of several QTc cut-off values: a cut-off value of 440 ms turned out to have 81% specificity, 89% sensitivity and 91% positive predictive accuracy for the diagnosis of Long QT Syndrome. Instead, specific cut-off values for gender (=440 ms for males and =460 ms for females) proved to be very specific (96%) but not very sensitive (72%).

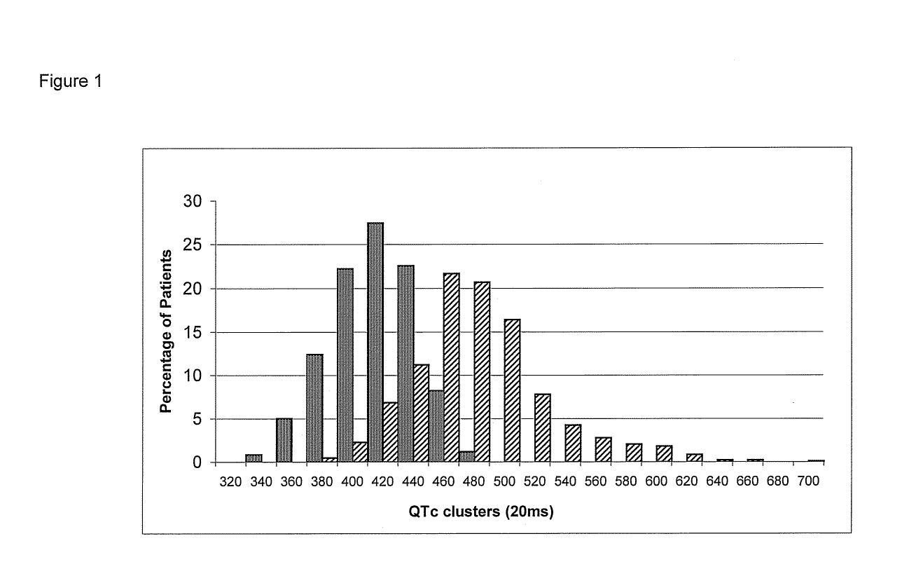

QTc duration correlates with the presence of genetic mutations: it is in fact decreasingly long for probands, family members carrying the disease at the genetic level and healthy family members (p<0.0001; Table 5). However the distribution of QTc values is very similar among individuals affected and unaffected by genetic mutations, even though the epidemiological data have shown that only 70% of subjects affected by the Syndrome has a prolonged QT interval (the overlap between the two populations is shown in FIG. 1). Therefore, it is demonstrated that the genetic diagnosis is an important tool for diagnosis of the disease at the presymptomatic stage. Moreover, since the type of underlying genetic defect affects the severity of the long QT Syndrome and the response to therapy, it is also evident the importance of molecular diagnosis for assessment of the arrhythmic risk and the following therapeutic choice (drugs, implantable defibrillator).

The data provided in the Table 3 and shown in FIG. 1, highlight the penetrance values of the disease (i.e. the percentage of carriers showing a prolongation of the QT interval in the surface electrocardiogram): from these data it is deduced that 30% of the carriers of at least one mutation according to the invention have a QT interval that is not different from normal. Therefore the effectiveness of a molecular screening for genotyping, like the one proposed here, is clear. Where necessary, such screening can be also coupled to a further investigation at the level of population screening, in order to identify genetic defects at birth, and/or identify iatrogenic long QT susceptibility, and/or screen competitive athletes and other populations in which the identification of a subclinical form of congenital long QT can prevent arrhythmic events, cardiac arrest and sudden cardiac death. This is made possible by the identification of a susceptibility to develop arrhythmias during exposure to trigger events (food, drugs etc) and the avoidance of conditions known to entail a higher risk, as for instance harmful life style habits, use of drugs and food/drinks contraindicated in subjects carrying such genetic defects.

The three levels of investigation in the method of the invention allow a significant saving of time and reagents: the first level of investigation is limited to the screening of about 70 mutations identified in Tables 2 A and 2B which are present in at least 40% of the patients that can be genotyped on the basis of mutations found so far. This way, a quick diagnosis is obtained that has limited costs and is nevertheless significant. Such analysis can be easily performed also in the forms of genetic screening at birth, screening for competitive athletes or screening for patients that need to be treated with drugs that can cause a prolongation of then QT interval. In fact, in all these categories of patients and sports persons, the analysis of the whole codifying portion of all disease genes is possible but is not used on a routine basis due to costs and excessive time length required.

The molecular method in its various embodiments makes use of well known methods for identification of mutations. Any method for detection of nucleotide mutations in a nucleic acid sequence can be used on the basis of the sequence information herein provided.

Among well known methods, the following are reported, although the list is not exhaustive: a) recognition of an enzymatic digestion pattern based for instance on the use of restriction enzymes by which the DNA fragment derived from the sample is selectively cut generating alternative patterns for mutated and “wild type” sequences, b) use of direct sequencing of nucleic acids, c) use of methods based on hybridization (or base pairing) with homologous or highly homologous sequences, d) use of the selective removal of specific sequences by methods of chemical or enzymatic breakage. The above techniques may or may not be used in association with steps of gene amplifications and may comprise, according to a particularly preferred embodiment, the use of solid platforms (microchip) with high/medium or low density binding of oligonucleotide probes (microarrays). Among hybridization-based methods beside the Southern Blotting technique, the following methods can also be used: Single Strand Conformation Polymorphism (SSCP Orita et al. 1989), clamped denaturing gel electrophoresis (CDGE, Sheffield et al. 1991), Denaturing Gradient Gel Electrophoresis (DGGE), heteroduplex analysis (HA, White et al. 1992), Chemical Mismatch Cleavage (CMC, Grompe et al., 1989), ASO (Allele Specific Oligonucleotides), RNase protection method (D B Thompson and J Sommercorn J. Biol. Chem., March 1992; 267: 5921-5926) and TCGE temporal gradient capillary electrophoresis Integrated platform for detection of DNA sequence variants using capillary array electrophoresis (Qingbo Li et al., Electrophoresis 2002, vol. 23, 1499-1511.

In the case of DNA direct sequencing (genes and ORFs) the primers used according to a preferred embodiment are those reported in Table 4. Insertions and deletions can be identified by methods well known in the art, such as RFLP (Restriction Fragment Length Polymorphism). Other methods are reported, for instance, in Sambrook et al. Molecular Cloning: a Laboratory Manual, Cold Spring Harbor Laboratory Press NY. USA, 1989, or in Human Molecular Genetics, ed Strachan T. and Read A. P., 2^{nd} ed. 1999, BIOS Scientific Publisher.

In a preferred embodiment the method of the invention, when based on nucleic acid hybridization, comprises the use of oligonucleotide probes derived from KCNQ1, KCNH2, SCN5A, KCNE1, KCNE2 genes, and comprising the nonanucleotides with sequence from SEQ ID NO: 11 to SEQ ID NO: 149 in the Sequence List or their complementary sequences, or oligonucleotides suitable to identify the mutations disclosed in the present invention.

Alternatively, the invention relates to oligonucleotides with wild type sequence, which are however suitable to distinguish, in a biological sample under appropriate conditions of hybridization stringency, the mutations or groups of mutations according to the invention because they cannot perfectly match with the mutated sequence present in the sample.

Considering that about 10-15% of subjects with iatrogenic (i.e. caused by drugs) torsions of the tip and sudden death show a Long QT Syndrome mutation as genetic substratum (Yang P et al Circulation. 2002 Apr. 23; 105(16):1943-8, Napolitano et al J Cardiovasc Electrophysiol. 2000 June; 11(6):691-6), one embodiment of the proposed method is definitively the identification of subjects with contraindication for drugs that prolong the QT interval by blocking the Ikr current (the so-called” pre-prescription genotyping”). These include antibiotics, prokinetic drugs, antipsychotic drugs, antidepressants, antiarrhythmic drugs and drugs belonging to other therapeutic classes.

Therefore, the method of the invention allows to avoid the risks associated with drug administration to subjects carrying the subclinical form of the Long QT Syndrome and to assess the sensitivity of a subject to the following drugs:

Albuterol, Alfuzosin, Haloperidol, Amantadine, Amiodarone, Amitriptyline, Amoxapine, Amphetamine/dextroamphetamine, AmpicillinArsenic trioxide, Atomoxetine, Azithromycin, Bepridil, Quinidine, Chloral hydrate, Chloroquine, Chlorpromazine, Ciprofloxacin, Cisapride, Citalopram, Clarithromycin, Clomipramine, Cocaine, Desipramine, Dextroamphetamine, Disopiramide, Dobutamine, Dofetilide, Dolasetron, Domperidone, Dopamine, Doxepin, Droperidol, Ephedrine, Epinephrine, Erythromycin, Felbamate, Fenfluramine, Phentermine, Phenylephrine, Phenylpropanolamine, Flecainide, Fluconazole, Fluoxetine, Foscarnet, Fosphenytoin, Galantamine, Gatifloxacin, Gemifloxacin, Granisetron, Halofantrine, Ibutilide, Imipramine, Indapamide, Isoproterenol, Isradipine, Itraconazole, Ketoconazole, Levalbuterol, Levofloxacin, Levomethadyl, Lithium, Mesoridazine, Metaproterenol, Methadone, Methylphenidate, Mexiletine, Midodrine, Moexipril/HCTZ, Moxifloxacin, Nicardipine, Norepinephrine, Nortriptyline, Octreotide, Ofloxacin, Ondansetron, Paroxetine, Pentamidine, Pimozide, Procainamide, Procainamide, Protriptyline, Pseudoephedrine, Quetiapine, Risperidone, Ritodrine, Roxithromycin, Salmeterol, Sertraline, Sibutramine, Solifenacin, Sotalol, Sparfloxacin, Tacrolimus, Tamoxifen, Telithromycin, Terbutaline, Thioridazine, Tizanidine, Trimethoprim-Sulfamethoxazole, Trimipramine, Vardenafil, Venlafaxine, Voriconazole, Ziprasidone, where the presence of at least one of the mutations identified in Table 1 or of one of the mutations identified in Table 2 indicates a sensitivity to one of the drugs mentioned above.

The assay can also be used to identify a possible susceptibility to develop arrhythmias during exposure to trigger events other than drugs (e.g. food). According to a preferred embodiment, the invention comprises kits for the realization of the diagnostic or prognostic methods according to each of the aspects described above. Therefore, said kits are obtained according to a preferred embodiment comprising at least one of the oligonucleotides in Table 1, having sequence from SEQ ID NO: 11 to SEQ ID NO: 149 (mutant sequences), and/or their complementary sequences, and optionally other reagents such as buffers, enzymes, etc. necessary to carry out the method of the invention. According to a preferred embodiment, the kit comprises a set of at least 2 oligonucleotides each including at least one of the following nonanucleotides (identified by the SEQ ID NO) or of their complementary sequences, where said nonanucleotides are chosen from:
KCNQ1 gene or locus: SEQ ID NO: 13 (L137), SEQ ID NO: 16 (R174P), SEQ ID NO: 17 (R190W), SEQ ID NO: 21 (I204M), SEQ ID NO: 24 (R231H), SEQ ID NO: 26 (V254L), SEQ ID NO: 27 (H258N), SEQ ID NO: 28 (H258R), SEQ ID NO: 29 (L262V), SEQ ID NO: 34 (V280E), SEQ ID NO: 39 (T322M), SEQ ID NO: 40 (P343L), SEQ ID NO: 41 (P343R), SEQ ID NO: 46 (R360T), SEQ ID NO: 55 (R518G), SEQ ID NO: 56 (R518P), SEQ ID NO: 59 (I567T), SEQ ID NO: 52, SEQ ID NO: 53;
KCNH2 gene or locus: SEQ ID NO: 67 (Y43C), SEQ ID NO: 69 (E58A), SEQ ID NO: 70 (E58G), SEQ ID NO: 71 (E58D), SEQ ID NO: 75 (delIAQ), SEQ ID NO: 85 (W412stop), SEQ ID NO: 88 (S428L), SEQ ID NO: 97 (G572D), SEQ ID NO: 98 (R852L), SEQ ID NO: 99 (D609H), SEQ ID NO: 106 (S660L), SEQ ID NO: 113 (S818P), SEQ ID NO: 79, SEQ ID NO: 80, SEQ ID NO: 110, SEQ ID NO: 111, SEQ ID NO: 125, SEQ ID NO: 126, SEQ ID NO: 128;
SCN5A gene or locus: SEQ ID NO: 133 (A413E), SEQ ID NO: 134 (A413T), SEQ ID NO: 140 (R1644C), SEQ ID NO: 142 (Y1767C).

Alternatively the kit comprises oligonucleotides suitable to detect a group of at least 20 of the mutations reported in Table 2, where said oligonucleotides are designed with methods or software well known in the art. According to a preferred embodiment, the kit to realize the diagnostic method of the invention comprises oligonucleotides suitable to detect the following mutations, identified, according to the numbering of codons or amino acids in the KCNQ1 gene:

L137F, where F is preferably identified with the corresponding codon in SEQ ID NO: 13; R174C and R174P, where P is preferably identified with the corresponding codon in SEQ ID NO: 16; G179S, R190W where W is preferably identified with the corresponding codon in SEQ ID NO: 17; and R190Q, I204M where M is preferably identified with the corresponding codon in SEQ ID NO: 20; R231C and R231H where H is preferably identified with the corresponding codon in SEQ ID NO: 24; D242N, V254L where L is preferably identified with the corresponding codon in SEQ ID NO: 26; and V254M, H258N where N is preferably identified with the corresponding codon in SEQ ID NO: 27; and H258R where R is preferably identified with the corresponding codon in SEQ ID NO: 28; R259C, L262V where V is preferably identified with the corresponding codon in SEQ ID NO: 29; G269D and G269S, S277L, V280E where E is preferably identified with the corresponding codon in SEQ ID NO: 34; A300T, W305S and W305stop, G314D and G314S, Y315C, T322M where M is preferably identified with the corresponding codon in SEQ ID NO: 39; G325R, A341E and A341V, P343C where C is preferably identified with the corresponding codon in SEQ ID NO: 40; and P343R where R is preferably identified with the corresponding codon in SEQ ID NO: 41; A344E, R360T where T is preferably identified with the corresponding codon in SEQ ID NO: 46; R518G where G is preferably identified with the corresponding codon in SEQ ID NO: 55; and R518P where P is preferably identified with the corresponding codon in SEQ ID NO: 56; and R518stop, R539W, 1567T where T is preferably identified with the corresponding codon in SEQ ID NO: 59; R591H, R594Q and, according to nucleotide numbering, with the mutations: 1514+1G>A, (SEQ ID NO: 52), 1513-1514delCA corresponding to SEQ ID NO: 53, with the mutation 921+1 G>A and with the mutation 921+2 T>C;
in the KCNH2 gene, according to the numbering of codons or amino acids: Y43C where C is preferably identified with the corresponding codon in SEQ ID NO: 67; E58A where A is preferably identified with the corresponding codon in SEQ ID NO: 69; and E58G where G is preferably identified with the corresponding codon in SEQ ID NO: 70; and E58D where D is preferably identified with the corresponding codon in SEQ ID NO: 71; and E58K, del82-84IAQ preferably identified with SEQ ID NO: 75; W412stop, S428L where L is preferably identified with the corresponding codon in SEQ ID NO: 88; R534C and R534L where L is preferably identified with the corresponding codon in SEQ ID NO: 91; L552S, A561T and A561V, G572C and G572D where D is preferably identified with the corresponding codon in SEQ ID NO: 97; R582C and R582L where L is preferably identified with the corresponding codon in SEQ ID NO: 98; G604S, D609H where H is preferably identified with the corresponding codon in SEQ ID NO: 99; and D609G, T613M, A614V, T6231, G628S, S660L where L is preferably identified with the corresponding codon in SEQ ID NO: 106; R752W, S818L, R823W and, according to nucleotide numbering, with the mutations: 453delC, 453-454insCC, 576delG (SEQ ID NO: 79), 578-582deICCGTG (SEQ ID NO: 80), G2398+3A>G (SEQ ID NO: 110), G2398+3A>T (SEQ ID NO: 111), 3093-3106del (SEQ ID NO: 125), 3093-3099del/insTTCGC (SEQ ID NO: 126), and 3100delC (SEQ ID NO: 128);
in the SCN5A gene: A413E where E is preferably identified with the corresponding codon in SEQ ID NO: 133; and A413T where T is preferably identified with the corresponding codon in SEQ ID NO: 134; T1304M, P1332L, 1505-1507delKPQ, R1623Q, R1644C where C is preferably identified with the corresponding codon in SEQ ID NO: 140; Y1767C where C is preferably identified with the corresponding codon in SEQ ID NO: 142, E1784K.

According to a different embodiment, the kit comprises a set of at least 20 mutations among those defined in Table 2 and optionally other reagents such as buffers, enzymes, etc. necessary to carry out the method of the invention.

TABLE 1
New mutations identified in probands.

| Gene | | | | | |
|---|---|---|---|---|---|
| Oligo IDN | | Nucleotide | Coding Effect (by aa* number) | Region | Type of mutation |
| | KCNQ1 | | | | |
| 11 | C CCG ACG GG | G136A | A46T | N-TERM | Missense |
| 12 | CGCTCTTAC | 151-152insT | L50fs + 233x | N-TERM | Insertion |
| 13 | C TGC TTC AT | C409T | L137F | S1 | Missense |
| 14 | C ATC AAG CA | G436A | E146K | S1-S2 | Missense |
| 15 | GTG GAC CGC | T518A | V173D | S2-S3 | Missense |
| 16 | GTC CCC CTC | G521C | R174P | S2-S3 | Missense |
| 17 | G GGG TGG CT | C568T | R190W | S2-S3 | Missense |
| 18 | CTG CCC TTT | G575C | R192P | S2-S3 | Missense |
| 19 | GCCCGAAGC | 584del | R195fs + 41X | S2-S3 | Deletion |
| 20 | C ATC CGTGA | G604C | D202H | S3 | Missense |
| 21 | TC ATG GTG G | C612G | I204M | S3 | Missense |
| 22 | GCC TTC ATG | C626T | S209F | S3 | Missense |
| 23 | C TGC ATG GG | G643A | V215M | S3 | Missense |
| 24 | ATC CAC TTC | G692A | R231H | S4 | Missense |
| 25 | ATG CCA CAC | T716C | L239P | S4 | Missense |
| 26 | C TCC TTG GT | G760T | V254L | S4-S5 | Missense |
| 27 | C ATC AAC CG | C772A | H258N | S4-S5 | Missense |
| 28 | ATC CGC CGC | A773G | H258R | S4-S5 | Missense |
| 29 | AG GAG GTG AT | C784G | L262V | S4-S5 | Missense |
| 30 | CACCTGTAC | 796del | T265fs + 22X | S5 | Deletion |
| 31 | CTG GAC CTC | G815A | G272D | S5 | Missense |
| 32 | TCTCGTACT | 828-830del | S277del | S5 | Deletion |
| 33 | TCC TGG TAC | C830G | S277W | S5 | Missense |
| 34 | TTT GAG TAC | T839A | V280E | S5 | Missense |
| 35 | GAC GAG GTG | C860A | A287E | S5-PORE | Missense |
| 36 | A GAT ACG CT | G904A | A302T | PORE | Missense |
| 37 | CAG GAC ACA | T923A | V308D | PORE | Missense |
| 38 | TAT GAG GAC | G947A | G316E | PORE | Missense |
| 39 | CAG ATG TGG | C965T | T322M | PORE-S6 | Missense |
| 40 | CTC CTA GCG | C1028T | P343L | S6 | Missense |
| 41 | CTC CGA GCG | C1028G | P343R | S6 | Missense |
| 42 | T GGC CCG GG | T1045C | S349P | S6 | Missense |
| 43 | C TCG CGG TT | G1048C | G350R | S6 | Missense |
| 44 | GGG TCT GCC | T1052C | F351S | S6 | Missense |
| 45 | GTGCAGCAG | 1067-1072del | QT 356-357del | C-TERM | Deletion |
| 46 | CAG ACG CAG | G1079C | R360T | C-TERM | Missense |
| 47 | GCA GAC TCA | C1115A | A372D | C-TERM | Missense |
| 48 | TGG ATG ATC | A1178T | K393M | C-TERM | Missense |
| 49 | GGGGGTGAC | 1291-1292insG | G430fs + 31x | C-TERM | Missense |
| 50 | GGGTGGACT | 1292-1293insG | V431fs + 31X | C-TERM | Insertion |
| 51 | AGACTGCTG | 1486-1487del | T495fs + 18X | C-TERM | Deletion |
| 52 | CACAATGAG | 1514 + 1 G > A | S504sp | C-TERM | Splice Error |
| 53 | CTCAGTGAG | 1513-1514del | S504fs + 9X | C-TERM | Deletion |
| 54 | GGCCACATT | 1538delC | T513fs + 78X | C-TERM | Deletion |
| 55 | C ATT GGA CG | C1552G | R518G | C-TERM | Missense |
| 56 | ATT CCA CGC | G1553C | R518P | C-TERM | Missense |
| 57 | CAG GAC CAC | G1643A | G548D | C-TERM | Missense |
| 58 | ATG GCG CGC | T1661C | V554A | C-TERM | Missense |
| 59 | TCC ACT GGG | T1700C | I567T | C-TERM | Missense |
| 60 | AAGCCTCAC | 1710delC | P570fs + 22X | C-TERM | Deletion |
| 61 | TG TTA ATC T | C1719A | F573L | C-TERM | Missense |
| 62 | ATCTCTCAG | 1725-1728del | S575fs + 16X | C-TERM | Missense |
| 63 | GAT CAC GGC | G1748A | R583H | C-TERM | Insertion |
| 64 | C AGC GAC AC | A1756G | N586D | C-TERM | Missense |
| 65 | CCCCCAGAG | 1893delC | P631fs + 33X | C-TERM | Deletion |
| 66 | GGGCCACAT | 1909delC | H637fs + 29X | C-TERM | Deletion |
| | KCNH2 | | | | |
| 67 | ATC TGC TGC | A128G | Y43C | N-TERM | Missense |
| 68 | TTC TGC GAG | G146A | C49Y | N-TERM | Missense |
| 69 | GCC GCG GTG | A173C | E58A | N-TERM | Missense |
| 70 | GCC GGG GTG | A173G | E58G | N-TERM | Missense |
| 71 | CC GAC GTG A | G174C | E58D | N-TERM | Missense |
| 72 | C GAC CTC CT | T202C | F68L | N-TERM | Missense |
| 73 | G CAC GGG CCG | G211C | G71R | N-TERM | Missense |
| 74 | CGC ACG CAG | C221T | T74M | N-TERM | Missense |
| 75 | GCAGGCAC | 244-252del | IAQ82-84del | N-TERM | Deletion |
| 76 | GATGATGGT | 308-310ins ATG | 103InsD | N-TERM | Insertion |
| 77 | TGTGCCCGT | 337-339del | V113del | N-TERM | Deletion |
| 78 | CGGTTCGCCG | 557- | A185fs + 143X | N-TERM | Deletion/Insertion |
| | | 566del/Ins + TTCGC | | | |
| 79 | CCGGGGCC | 576delG | G192fs + 7X | N-TERM | Deletion |
| 80 | GGGGGTGGT | 578-582del | G192fs + 135X | N-TERM | Deletion |
| 81 | GCCCCCGGC | 735-6InsCC | P245fs + 114X | N-TERM | Insertion |
| 82 | ATCGTCCCG | C751T | P251S | N-TERM | Missense |
| 83 | G CTG TAG G | C1171T | Q391X | N-TERM | Nonsense |
| 84 | C GTG TCG GAC | G1229C | W410S | S1 | Missense |
| 85 | G GAC TGA CTC | G1235A | W412X | S1 | Nonsense |
| 86 | C ACA CAC TAC | C1277A | P426H | S1-S2 | Missense |
| 87 | A CCC CAC TCG | T1279C | Y427H | S1-S2 | Missense |
| 88 | C TAC TTG GCT | C1283T | S428L | S1-S2 | Missense |
| 89 | C GTG TAC ATC | G1378T | D460Y | S2 | Missense |
| 90 | C ATC CAC ATG | G1501C | D501H | S3 | Missense |
| 91 | G GTG CTC GTG | G1601T | R534L | S4 | Missense |
| 92 | CGGATCGCT | 1613-1619del | R537fs + 24X | S4 | Deletion |
| 93 | GCC TCC ATC | G1697C | C566S | S5 | Missense |
| 94 | TGCATTGGT | 1701delC | I567fs + 26X | S5 | Deletion |
| 95 | C ATC CGG TAC | T1702C | W568R | S5 | Missense |
| 96 | C GCC GTC GC | A1711G | I571V | S5 | Missense |
| 97 | C ATC GAC AA | G1715A | G572D | S5 | Missense |
| 98 | TCA CTC ATC | G1745T | R582L | S5-PORE | Missense |
| 99 | AAG CAC AAG | G1825C | D609H | S5-PORE | Missense |
| 100 | GCG TTC TAC | C1843T | L615F | PORE | Missense |
| 101 | GC AGG CTC A | C1863G | S621R | PORE | Missense |
| 102 | CCC GCC AGC | G1877C | G626A | PORE | Missense |
| 103 | TCA GAC AAG | G1911C | E637D | PORE-S6 | Missense |
| 104 | C TGC TTC AT | G1930T | V644F | S6 | Missense |
| 105 | ATC TGC GGC | T1967G | F656C | S6 | Missense |
| 106 | TG TTG GCC A | C1979T | S660L | S6 | Missense |
| 107 | CAG CCC CTC | G2087C | R696P | S6-CNBD | Missense |
| 108 | TGACGAGTG | 2164-2181dup | E722-D727 | S6-CNBD | Duplication |
| 109 | CTTCCAGGG | 2231delG | F743fs + 12X | S6-CNBD | Deletion |
| 110 | GGGTGTGGG | G2398 + 3A > G | L799sp | CNBD | Splice Error |
| 111 | GGGTTTGGG | G2398 + 3A > T | L799sp | CNBD | Splice Error |
| 112 | CCTGTGTAT | G2398T | G800W | CNBD | Missense |
| 113 | AAG CCG AAC | T2452C | S818P | CNBD | Missense |
| 114 | T GGC TAG TC | A2494T | K832X | CNBD | Nonsense |
| 115 | TTC CAC CTG | A2581C | N861H | C-TERM | Missense |
| 116 | GGGTGCAAC | 2638-2648del | G879fs + 35X | C-TERM | Deletion |
| 117 | TCCGACGGA | 2676-2682del | R892fs + 79X | C-TERM | Deletion |
| 118 | CCGGCCGGG | 2732-2766del | P910 + 16X | C-TERM | Deletion |
| 119 | GGCGGGCCG | 2738-2739insCGGGC | A913fs + 62X | C-TERM | Insertion |
| 120 | GGGGCCGTG | 2775delG | G925fs + 47X | C-TERM | Deletion |
| 121 | CG TGA GGG G | G2781A | W927X | C-TERM | Nonsense |
| 122 | CCCGGGTGG | 2895-2905del | G965fs + 148X | C-TERM | Deletion |
| 123 | CCG CTG GGT | C2903T | P968L | C-TERM | Missense |
| 124 | CGATGACCCGC | C3045A | C1015X | C-TERM | Nonsense |
| 125 | CCCGGGGCG | 3093-3106del | G1031fs + 86X | C-TERM | Deletion |
| 126 | GGGTTCGCC | 3093- | G1031fs + 20X | C-TERM | Deletion/Insertion |
| | | 3099del/insTTCGC | | | |
| 127 | GGCGCCCCG | 3099delG | R1033 + 22X | C-TERM | Missense |
| 128 | GCGGCCCGG | 3100delC | P1034 fs + 63X | C-TERM | Deletion |
| 129 | CAGGTGGAG | 3154delC | R1051fs + 4X | C-TERM | Deletion |
| 130 | CCCCACCCT | 3304InsC | P1101fs + 16X | C-TERM | Insertion |
| 131 | CCCACGACG | 3397-3398del | T1133fs + 135X | C-TERM | Deletion |
| | SCN5A | | | | |
| 132 | CTG TAC AGA | C3457T | H1153Y | C-TERM | Missense |
| 133 | GGTCGAA ATG | C1238A | A413E | IS6 | Missense |
| 134 | GGTCACAAT | G1237A | A413T | IS6 | Missense |
| 135 | TGCC GAG GG | C1717G | Q573E | I-II | Missense |
| 136 | TCCC AGA AC | G1735A | G579R | I-II | Missense |
| 137 | AAC CAT CTC | G2066A | R689H | I-II | Missense |
| 138 | T GCC ACG AAG | T4493C | M1498T | III-IV | Missense |
| 139 | TC TTC CCA GTC | G4877C | R1626P | IV-S4 | Missense |
| 140 | GATC TGC ACG | C4930T | R1644C | IV-S4 | Missense |
| 141 | C AAC GTC GG | A4978G | I1660V | IV-S5 | Missense |
| 142 | ATG TGC ATT | A5300G | Y1767C | IV-S6 | Missense |
| 143 | CACC AAG CC | G5360A | S1787N | C-TERM | Missense |
| 144 | GAG GGC GAC | A5369G | D1790G | C-TERM | Missense |
| 145 | TTC CAC AGG | G5738A | R1913H | C-TERM | Missense |
| | KCNE1 | | | | |
| 146 | CCC CAC AGC | G107A | R36H | S1 | Missense |
| 147 | GGA TGC TTC | T158G | F53C | S1 | Missense |
| | KCNE2 | | | | |
| 148 | GTGGTG ATG | A166G + 169InsATG | M56V + M57ins | S1 | Missense/Insertion |
| 149 | CTGTAGGTG | 156-161del | 52-54del.YLM > X | S1 | Deletion |

TABLE 2A
Mutations found in at least 40% of probands.
MISSENSE MUTATIONS AND IN-FRAME INSERTIONS OR DELETIONS
NUMBERING REFERS TO THE AMINO ACID SEQUENCE

| KCNQ1 | KCNH2 | SCN5A |
|---|---|---|
| L137 → (Z-L) F (SEQ ID NO: 13) | Y43 → (Z -Y) C (SEQ ID NO: 67) | A413 → (Z -A) E (SEQ ID NO: 133), T |
| | | (SEQ ID NO: 134) |
| R174 → (Z-R) C¹, P (SEQ ID NO: 16) | E58 → (Z -E) A, G, D (SEQ ID NO: | T1304 → (Z -T) M³ |
| | 69, 70, 71), K² | |
| G179 → (Z-G) S⁴ | del 82-84: IAQ (SEQ ID NO: 75) | P1332 V (Z -P) L⁵ |
| R190 → (Z-R) W (SEQ ID NO: 17), Q¹ | W412 → (Z -W); X (SEQ ID NO: 85) | del 1505-1507: KPQ⁶ |
| I204 → (Z -I) M (SEQ ID NO: 21) | S428 → (Z -S) L (SEQ ID NO: 88), X⁷ | R1623 → (Z -R) Q⁸ |
| R231 → (Z -R) C⁹, H (SEQ ID NO: 24) | R534 → (Z -R) C¹⁰ | R1644 → (Z -R) C (SEQ ID NO: 140), H⁴ |
| D242 → (Z -D) N¹⁰ | L552 → (Z -L) S⁴ | Y1767 → (Z -Y) C (SEQ ID NO: 142) |
| V254 → (Z -V) L (SEQ ID NO: 26), M¹ | A561 → (Z -A) T⁴, V⁴ | E1784 → (Z -E) K⁴ |
| H258 → (Z-H) N (SEQ ID NO: 27), R (SEQ | G572 → (Z -G) C⁴, D (SEQ ID NO: 97) | |
| ID NO: 28) | | |
| R259 → (Z -R) C¹¹ | R582 → (Z -R) C¹², L (SEQ ID NO: 98) | |
| L262 → (Z -L) V (SEQ ID NO: 29) | G604 → (Z -G) S⁴ | |
| G269 → (Z -G) D¹, S¹³ | D609 → (Z -D) H (SEQ ID NO: 99), G¹⁶ | |
| S277 → (Z -S) L¹⁴ | T613 → (Z -T) M⁴ | |
| V280 → (Z -V) E (SEQ ID NO: 34) | A614 → (Z -A) V⁷ | |
| A300 → (Z -A) T¹⁵ | T623 → (Z -T) I¹⁶ | |
| W305 → (Z -W) S¹⁷, X¹⁸ | G628 → (Z -G) S⁴ | |
| G314 → (Z -G) D¹⁹, S¹ | S660 → (Z -S) L (SEQ ID NO: 106) | |
| Y315 → (Z -Y) C²⁰ | R752 → (Z -R) W⁴ | |
| T322 → (Z -T) M (SEQ ID NO: 39) | S818 → (Z -S) L²¹, P (SEQ ID NO: 113) | |
| G325 → (Z -G) R¹ | R823 → (Z -R) W⁴ | |
| A341 → (Z -A) E⁴, V⁴ | | |
| P343 → (Z -P) L, R (SEQ ID NO: 40, | | |
| 41) | | |
| A344 → (Z -A) E¹⁶ | | |
| R360 → (Z -R) T (SEQ ID NO: 46) | | |
| R518 → (Z -R) G, P (SEQ ID NO: 55, | | |
| 56), X⁴ | | |
| R539 → (Z -F) W²² | | |
| I567 → (Z -I) T (SEQ ID NO: 59) | | |
| R591 → (Z -R) H²³ | | |
| R594 → (Z -R) Q⁴ | | |

TABLE 2B
SPLICING AND FRAMESHIFTS MUTATIONS (NUMBERING
REFERS TO THE NUCLEOTIDE SEQUENCE)

| KCNQ1 | KCNH2 |
|---|---|
| 1514 + 1 G > A (SEQ ID NO: 52) | 453delC²⁴; 453-454insCC¹⁶ |
| 1513-1514delCA (SEQ ID NO: 53) | 576delG (SEQ ID NO: 79) |
| 921 + 1 G > A⁴ | 578-582del CCGTG (SEQ ID NO: 80) |
| 921 + 2 T > C⁴ | G2398 + 3A > G (SEQ ID NO: 110) |
| | G2398 + 3A > T (SEQ ID NO: 111) |
| | 3093-3106del (SEQ ID NO: 125) |
| | 3093-3099del/insTTCGC (SEQ ID NO: 126) |
| | 3100delC (SEQ ID NO: 128) |

### Legend to Tables 1 and 2.

### Table 1.

### Mutation Abbreviations:

del: deletion. 796del indicates that the nucleotide at position 796 is deleted. When more than one nucleotide is deleted, as for instance in 828-830 (SEQ ID NO: 32), all the intervening nucleotides and the extreme nucleotides are deleted (e.g. 828, 829, 830). The deleted nucleotide/nucleotides can be also specified (e.g. in SEQ ID NO: 129, 3154delC indicates that the cytosine at position 3154 is deleted).
ins: insertion. The explanation is similar to del.

Generally, the nucleotide or amino acid number refers to the residue (nucleotide or amino acid) affected by the mutation. However, for frame shift mutations (fs), the amino acid residue indicated with the one-letter code and the number corresponding to its position in the protein sequence, corresponds to the last residue identical to wild type.

Moreover, for frame-shift mutations, that easily generate a stop codon upstream of the natural stop codon, it is also indicated the number of amino acids out of frame (different from the natural protein sequence) that follow the last wild type amino acid (for instance, R195fs+41X, SEQ ID NO: 19, indicates that the nucleotide mutation generates a frame-shift due to which Arg at position 195 of the KCNQ1 gene is the last wild type amino acid, followed by a tail of 41 amino acids different from wild type).

Coding for splicing (sp) errors: the number indicates the last nucleotide of the exon; the number after the +sign indicates the position, relative to this nucleotide, of the intronic base that is substituted. For instance, 921+2 T>C indicates a T to C substitution of the second intronic base after the last exonic nucleotide at position 921.

### Table 2A

Z: any amino acid; (Z-W): any amino acid but W. Therefore, the detection of the mutation refers to the codon identified by the number: Detection may refer to any codon different from wild type (e.g. for KCNQ1, the detection of R518 refers to any codon not encoding the wild type amino acid isoleucine, and preferably refers to any codon encoding glycine or proline, even more preferably refers to glycine and proline codons as identified by SEQ ID NO: 55 and SEQ ID NO: 56).

### Table 2A and 2B:

X.: STOP codon. Other abbreviations and symbols have the same meaning as in Table 1.
Numbering refers to the number of the wild type amino acid (Table 2A) or nucleotide (Table 2B) as reported in the Sequence List.
Superscript numbers refer to references for mutations already described. The mutations identified in the present invention are characterized by the corresponding identification n° (SEQ ID NO) of Table 1.
Additional references for the symbols used are reported in: Antonarakis S E. Recommendations for a nomenclature system for human gene mutations. Nomenclature Working group. Hum. Mutat., 1998; 11:1-3.

### REFERENCES FOR TABLE 2

1. Donger, C, et al. Circulation. 1997; 96:2778-2781.
2. Lupoglazoff, J M, et al. Circulation. 2001; 103:1095-1101.
3. Wattanasirichaigoon, D, et al. Am J Med Genet. 1999; 86:470-476.
4. Splawski, I, et al. Circulation. 2000; 102:1178-1185.
5. Kehl, H G, et al. Circulation. 2004; 109:e205-e206.
6. Wang, Q, et al. Cell. 1995; 80:805-811.
7. Priori, S G, et al. Circulation. 1999; 99:529-533.
8. Kambouris, N G, et al. Circulation. 1998; 97:640-644.
9. Lupoglazoff, J M et al. J Am Coll Cardiol. 2004; 43:826-830.
10. Itoh, T, et al. Hum. Genet. 1998; 102:435-439.
11. Kubota, T, et al. J Cardiovasc Electrophysiol. 2000; 11:1048-1054.
12. Jongbloed, R J, et al. Hum. Mutat. 1999; 13:301-310.
13. Ackerman, M J, et al N. Engl. J. Med. 1999; 341:1121-1125.
14. Liu, W, et al. Hum. Mutat. 2002; 20:475-476.
15. Priori, S G, et al. Circulation. 1998; 97:2420-2425.
16. Tester, D J, et al. Heart Rhythm. 2005; 2:507-517.
17. Neyroud, N, et al. Eur. J. Hum. Genet. 1998; 6:129-133.
18. Chen, S et al. Clin Genet. 2003; 63:273-282.
19. Choi, G, et al. Circulation. 2004; 110:2119-2124.
20. Napolitano, C et al. J. Cardiovasc. Electrophysiol. 2000; 11:691-696.
21. Berthet, M, et al. Circulation. 1999; 99:1464-1470.
22. Chouabe, C, et al. Cardiovasc Res. 2000; 45:971-980.
23. Neyroud, N, et al. Circ. Res. 1999; 84:290-297.
24. Swan, H, et al J. Am. Coll. Cardiol. 1999; 34:823-829.

TABLE 3

| | N | QTc (ms) | Mean | IQR | % penetrance |
|---|---|---|---|---|---|
| LQT1 | 450 | 465 ± 41 | 461 | 440-488 | 64** |
| LQT2 | 279 | 486 ± 48* | 477 | 455-511 | 81# |
| LQT3 | 63 | 489 ± 49* | 481 | 460-515 | 83# |
| LQT5 | 20 | 447 ± 36 | 439 | 424-467 | 40 |
| LQT6 | 5 | 434 ± 24 | 425 | 414-459 | 40 |

QT values in ms.
IQR = Inter Quartile Range in ms;
*p < 0.005 vs. KCNQ1/KCNE1;
**p < 0.04 vs. KCNE1;
#p < 0.001 vs. KCNQ1/KCNE1/KCNE2

TABLE 4

Sequencing primers (SEQ ID NO: 150-212)

| | | | |
|---|---|---|---|
| GENE | PRIMER ID | SEQUENCE | Length |

| | | | |
|---|---|---|---|
| KCNQI | KV11.1 | cactcaaggccgagcctgcct | 21 |
| ″ | KV5NA | gccccacaccatctccttcg | 20 |
| ″ | KV6NI | taccctaacccgggccac | 18 |
| ″ | KVDFn | gaggagaagtgatgcgtgtc | 20 |
| ″ | KVDRn | ggcaggacctgggcaccctc | 20 |
| ″ | KV1A1F | cttcgctgcagctcccggtg | 20 |
| ″ | KV1A2R | acgcgcgggtctaggctcac | 20 |
| ″ | KK2F | gactgccgtgtccctgtcttg | 21 |
| ″ | KK2R | gccatgccttcagatgctacg | 21 |
| ″ | KK12Rn | ctgagggcaggaaggctcag | 20 |
| ″ | KK14F1 | ctgtctgtcccacagacgac | 20 |
| ″ | KK14Rn | ctgggcccagagtaactgac | 20 |
| ″ | KK15Fn | cggcccaccccagcacttggc | 21 |
| ″ | KK15Rn | gaaccaccgcaggccggcgcg | 21 |
| ″ | KK16Fn | cgtctgcctttgtccccg | 18 |
| ″ | KK16Rn | cactcttggcctcccctc | 18 |
| KCNE1 | MINK F | ctgcagcagtggaccctta | 19 |
| ″ | MINK 1R | agcttcttggagcggatgta | 20 |
| ″ | MINK 2F | gtcctcatggtactgggatt | 20 |
| ″ | MINK R | tttagccagtggtggggtt | 19 |
| KCNE2 | MINK2-F2 | ccgttttcctaaccttgttcgcct | 24 |
| ″ | MINK2-R2 | gccacgatgatgaaagagaacattcc | 26 |
| ″ | MINK2-F3 | gtcatcctgtacctcatggtgat | 23 |
| ″ | MINK2-R3 | tggacgtcagatgttagcttggtg | 24 |
| SCN5A | SCN2.1Fnew | ccc tgc tct ctg tcc ctg | 21 |
| | | ggc | |
| ″ | SCN2.1Rnew | gca gcc cct ctc ggc tct | 20 |
| | | cc | |
| ″ | SCN2.2Fnew | cat ggc aga gaa gca agc | 21 |
| | | ccg | |
| ″ | SCN6Fnew | cct cct ctg act gtg tgt | 22 |
| | | ctc c | |
| ″ | SCN10Fnew | cca gtg agg gtg acc tct | 21 |
| | | gcc | |
| ″ | SCN10Rnew | ggc tta gag gct cct cgg | 21 |
| | | tgg | |
| ″ | SCN16F | gag cca gag acc ttc aca | 21 |
| | | agg | |
| ″ | SCN17.1Fnew | gct tgg cat ggt gca gtg | 24 |
| | | cct tgg | |
| ″ | SCN17.1Rnew | gag gca cct tct ccg tct | 22 |
| | | ctg g | |
| SCN5A | SCN20Fnew | cat tag atg tgg gca ttc | 24 |
| | | aca ggc | |
| ″ | SCN20Rnew | cca gcc gtc cct gcc aca | 21 |
| | | acc | |
| ″ | SCN21Fnew | ggt cca ggc ttc atg tcc | 21 |
| | | acc | |
| ″ | SCN21Rnew | ggc aat ggg ttt ctc ctt | 22 |
| | | cct g | |
| ″ | SCN22Fnew | ggg gag ctg ttc cca tcc | 22 |
| | | tcc c | |
| ″ | SCN22Rnew | cgc ctc cca ctc cct ggt | 21 |
| | | ggg | |
| ″ | SCN23.1F | ttg aaa agg aaa tgt gct | 23 |
| | | ctg gg | |
| ″ | SCN23.1R | ttg ttc acg atg gtg tag | 22 |
| | | ttc a | |
| ″ | SCN23.2F | cca gac aga ggg aga ctt | 21 |
| | | gcc | |
| ″ | SCN25Fnew | ccc agc ctg tct gat ctc | 22 |
| | | cct g | |
| ″ | SCN25Rnew | cca ccc tac cca gcc cag | 21 |
| | | tgg | |
| KCNH2 | HM1F | catgggctcaggatgccggt | 20 |
| ″ | KCNH2-1R | cattgactcgcacttgccgacg | 22 |
| ″ | H2F | cgctcacgcgcactctcctc | 20 |
| ″ | KH2R | ttgaccccgcccctggtcgt | 20 |
| ″ | H3F | ccactgagtgggtgccaaggg | 21 |
| ″ | H3R | gagaccacgaacccctgagcc | 21 |
| ″ | H4.1F | cccacgaccacgtgcctctcc | 21 |
| ″ | H8R | gcctgccacccactggcc | 18 |
| ″ | KH9F | atggtggagtagagtgtgggtt | 22 |
| ″ | KH9R | agaaggctcgcacctcttgag | 21 |
| ″ | KH10F | gagaaggtgcctgctgcctgg | 21 |
| ″ | KH10R | acagctggaagcaggaggatg | 21 |
| ″ | H11F | ggcaggagagcactgaaagggc | 22 |
| ″ | H11R | ggtaaagcagacacggcccacc | 22 |
| ″ | H12A F | gttctcctcccctctctgaggc | 22 |
| ″ | H12B R | gggtagacgcaccaccgctgc | 21 |
| ″ | H13F | gcagcggtggtgcgtctaccc | 21 |
| ″ | H13R | gacctggaccagactccagggc | 22 |
| ″ | H14R | gggtacatcgaggaagcagg | 20 |

### EXPERIMENTAL PART

### Methods

Probands and their relatives were subjected to genetic analysis by molecular screening of the coding regions of genes associated with LQTS (Romano Ward variant). The diagnosis of probands was based on conventional clinical criteria (personal clinical history, evaluation of the QT interval by standard 12-lead ECG, Holter recording, with a cycloergometer exercise test).

Relatives were evaluated by genetic analysis independently from the diagnosis based on the clinical phenotype.

Methods for sample analysis: the entire coding regions of KCNQ1, KCNH2, SCN5A, KCNE1 and KCNE2 genes were examined in a sample of 1621 subjects belonging to 430 families, using primers designed from intronic regions close to splicing sites (Table 4).

The population study comprised 430 LQTS probands with RWS and 1115 members of their families, and their data were collected by the Molecular Cardiology Laboratories of the Maugeri Foundation.

An informed consent was obtained from all subjects undergoing molecular analysis and, in the case of minors, from their legal tutors.

A group of 75 genotyped probands were used to verify the non-familial mutations identified in the previously examined population.

The study was approved by the Institutional Review Board of the Maugeri Foundation according to IRB regulations for all study subjects.

### Statistical Analysis

Statistical analyses, when performed, were carried out with the SPSS Statistical Package (v. 12.01). The Kolmogorov-Smirnov test was used to determine the normal distribution of variables. Parametric tests (unpaired t-test and ANOVA with Bonferroni multiple-comparison correction) were used to compare normally distributed variables; instead the Kruskal-Wallis and the Mann-Whitney tests were used for not normally distributed variables. Chi-square test or Fisher exact test has been used for categorical variables. The data obtained are the mean±standard deviation (SD). For data that do not follow a normal distribution, the median and the interquartile range (25% and 75%) are reported.

### Genotyping

Molecular analysis was performed on genomic DNA extracted from peripheral lymphocytes using methods well known in the art. The entire “open reading frames” of KCNQ1, KCNH2, SCN5A, KCNE1 and KCE2 genes were amplified by PCR with primers designed from intronic sequences flanking the exons (“exon-flanking intronic primer”). Primers are well known in the art or listed in Table 4. Amplicons were analysed by Denaturing High Performance Liquid Chromatography (DHPLC, Wave® Transgenomics) Each amplicon was subjected to DHPLC at least at two different temperatures, depending on the “melting” profile of the amplicon. When an abnormal chromatogram was detected, the amplicon was sequenced by a 310 Automated Genetic Analyzer® (Applied Biosystems) and/or cloned by PCR in a plasmid vector (Topo® cloning, Invitrogen). All DNA sequence variations that were absent in 400 control subjects (corresponding to 800 chromosomes) were defined as mutations.

### Example 1

### Clinical Characterization of the Sample

ECGs and clinical data were collected and analyzed in blind relative to the genetic status of the samples. QTc (QTc=QT/vRR) data were measured using the ECG recorded during the first visit.

The demographic characteristics of the sample are shown in the following table.

TABLE 5

| | | Family members | | |
|---|---|---|---|---|
| | | Genetically | Genetically not | |
| | Probands | affected | affected | P-value |
| N | 310 | 521 | 594 | |
| Males (%) | 147 (47) | 231 (44) | 281 (47) | |
| Age, years (mean) | 21 ± 20 (16) | 33 ± 20 (35) | 29 ± 19 | <0.002** |
| QTc* (mean) | 495 ± 49 (490) | 461 ± 40 (458) | 406 ± 27 (408) | <0.001** |

*In ms.
**Post-hoc analysis between groups.
QTc duration and occurrence by genotype.

The QTc value in the various populations examined has been reported in Table 3. As can be deduced from the table, in the mutation carriers the QTc was 474+46 msec (median value: 467 msec, IQR: 444-495 msec) while, among healthy family members, it was 406±27 msec (median value: 409 msec, IQR 390-425 msec). Incomplete penetrance was defined as the percentage of mutation carriers having a QTc longer than normal (e.g., QTc=440 ms for males and =460 ms for females).

The mean penetrance of the disease in the study population turned out to be 70%, but decreased to 57% among family members carrying the mutation. Patients with LQT2 and LQT3 showed a higher penetrance compared to patients with LQT1 and LQT5, whereas penetrance for patients with LQT6 could not be determined due to the low number (see Table 5).

QTc duration was decreasingly long for probands, family members carrying the disease at the genetic level and healthy family members (p<0.0001; Table 5). However the distribution of QTc values was very similar between subjects affected by genetic mutations and subjects without mutations. (Instead, the QTc and the penetrance of LQTS were significantly different between carriers of multiple mutations (n=26) and family members with only one mutation (n=49): QTc 495±58 msec, IQR 450-523 msec versus 434+31 msec, IQR: 411-452 msec, p<0.001; 30%, 15/49 versus 77% 20/26; p<0.0001).

The analysis of 1411 subjects (296 probands, 521 genetically affected and 594 genetically unaffected family members) to determine the sensitivity and the specificity of different QTc cut-off intervals in individuals carrying genetic mutations, revealed that the best cut-off value for sensitivity and specificity is a QTc value=440 ms (81% specificity, 89% sensitivity and 91% positive predictive accuracy for diagnosis). Specific cut-off values for gender (=440 ms for males and =460 ms for females) proved to be very specific (96%) but poorly sensitive (72%).

### Example 2

### Genetic Characterization of the Sample

Genetic analysis of the sample revealed that 310/430 (72%) of the probands and 521 family members were carriers of 235 different mutations (139 of which were novel) that can determine the LQTS Syndrome.

From these 310 probands, the genetic analysis was extended to a total of 1115 family members: a mutation was found in 521 of them, while 594 were found to be healthy. In total, the study revealed 831 carriers of a mutation predisposing to LQTS symptoms and 594 healthy family members.

The mutation rate in the different genes was distributed as follows: 49% KCNQ1; 39% KCNH2; 10% SCN5A; 1.7% KCNE1; 0.7% KCNE2. About 90% of genotyped patients had mutations in KCNQ1 and KCNH2 genes, while 44% of the probands carried common mutations. These statistics were verified again with an independent set of 75 genotyped probands (FIG. 1).

All the mutations identified and characterized for the first time in the present invention are reported in Table 1.

Two-hundred-ninety-six probands carried heterozygous mutations and 14 (4.5%) carried more than one genetic defect. Twelve probands turned out to be heterozygous for 2 (n=11) or 3 (n=1) combinations of mutations, 2 turned out to be homozygous RWS patients. In the group of 296 probands with a single genetic defect, KCNQ1mutations were most represented (n=144; 49%), followed by KCNH2 mutations (n=115; 39%), SCN5A mutations (n=30; 10%), KCNE1 mutations (n=5; 1.7%) and at last KCNE2 mutations (n=2; 0.7%).

In conclusion, 98% of the genotypic mutations detected in LQTS probands were identified in KCNQ1, KCNH2 and SCN5A genes. Twenty-nine of 247 probands, whose parents were both available for genetic analysis, turned out to be carriers of sporadic mutations (12%).

Overall, 235 different mutations were identified, of which 139 (KCNQ1 n=56, KCNH2 n=67, SCN5A n=13, KCNE1 n=2, KCNE2 n=2) were identified in this study for the first time. Missense mutations accounted for 72% (170/235) of the genetic defects. The remaining 28% comprised small intragenic deletions (n=33; 14.1%), splice errors (n=6; 2.7%), non-sense mutations (n=12; 5.1%), insertions (n=11; 4.7%), duplications or insertions/deletions (n=3; 1.4%). The most frequently mutated codons (hot-spots) were: in KCNQ1: codon 190 (n=12), 231 (n=4) 254 (n=4), 269 (n=4), 277 (n=5), 314 (n=4), 341 (n=6), 344 (n=9) and codons 561 (n=7), 572 (n=4) and 628 (n=7) in KCNH2, 1332 (n=5) and 1784 (n=3) in SCN5A. In total, 74/296 (25%) of the probands were genotyped based on hot-spot mutations and 129/296 of the probands (44%) carried one of the non-private mutations reported in Table 2 (i.e. mutations identified in more than one family).

### Intra-Locus Variability

The distribution of mutations in the protein coding regions of the various LQTS genes, using the subdivision in regions already reported in other studies (e.g. Splawski I. et al. Circulation, 2000 102:1178-1185), was the following: mutations in the “pore region” and in the transmembrane region were identified in 61% of the patients, while in 32% of the patients the mutation was in a C-terminal region; only 7% of the patients carried a N-terminal mutation.

### Annex 1

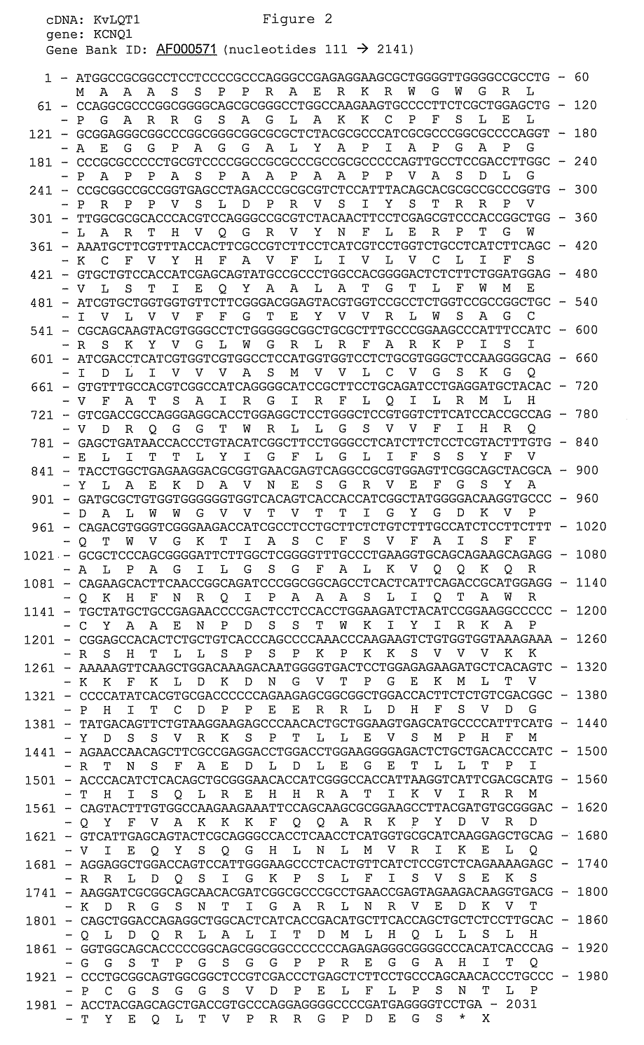

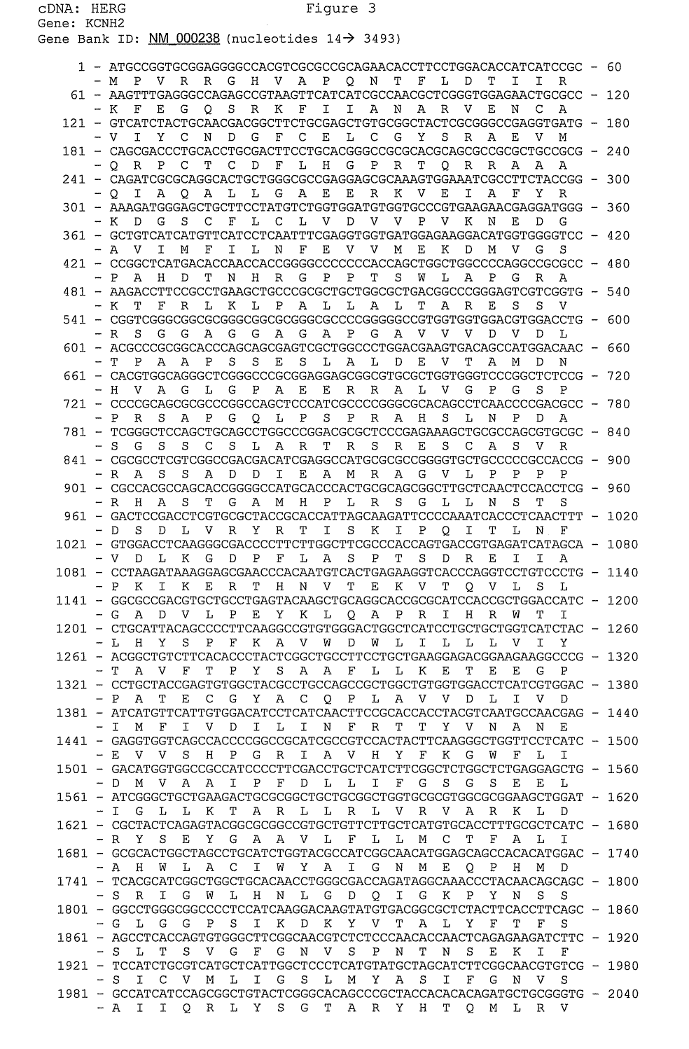

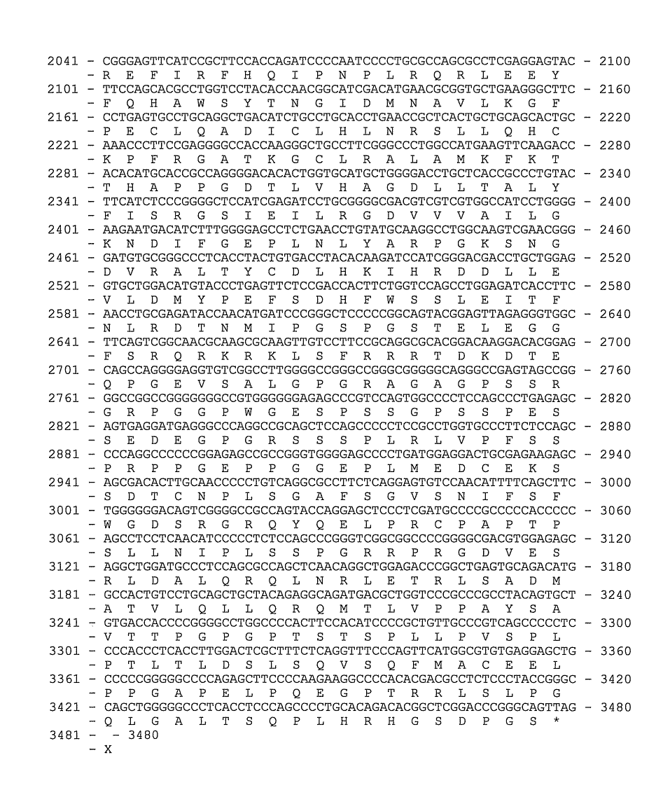

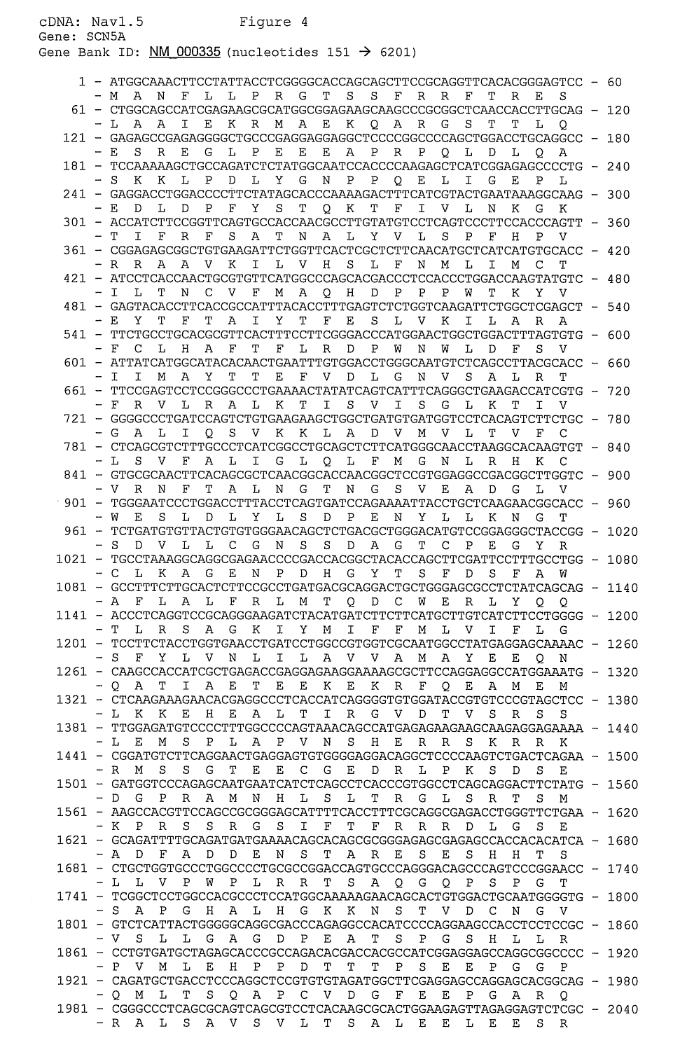

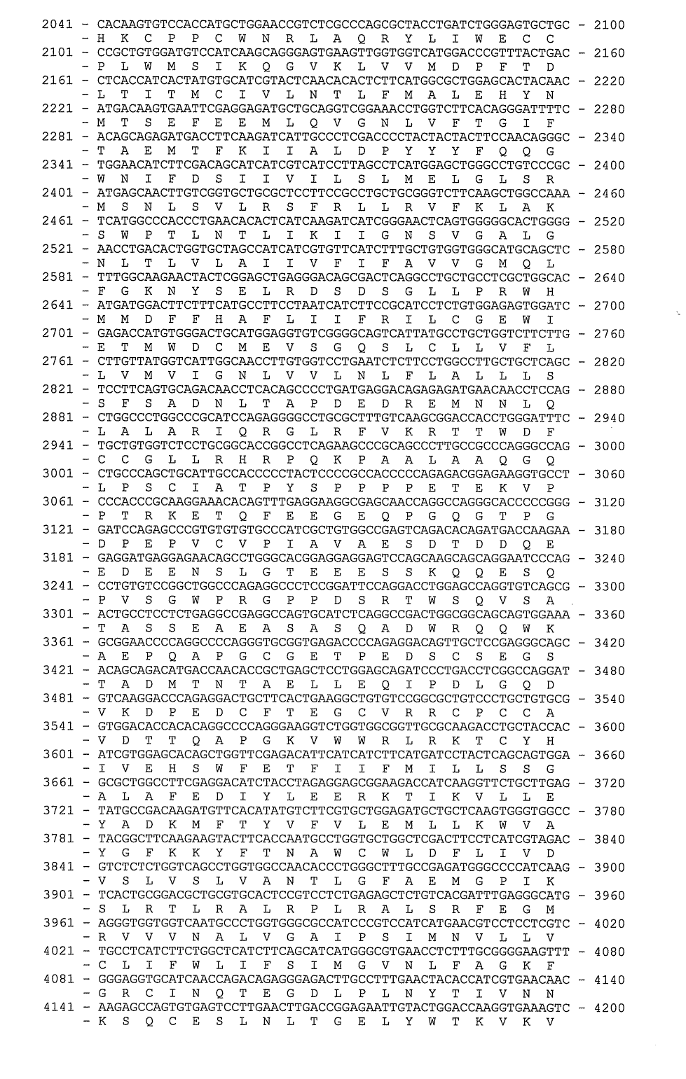

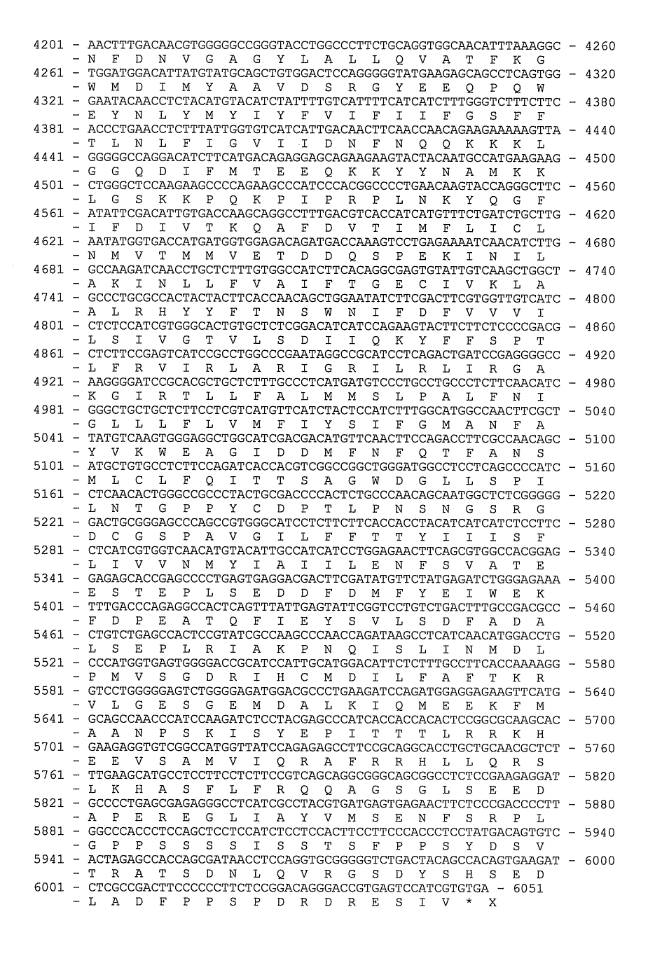

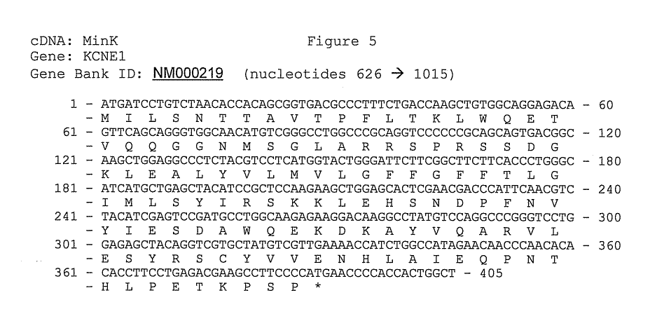

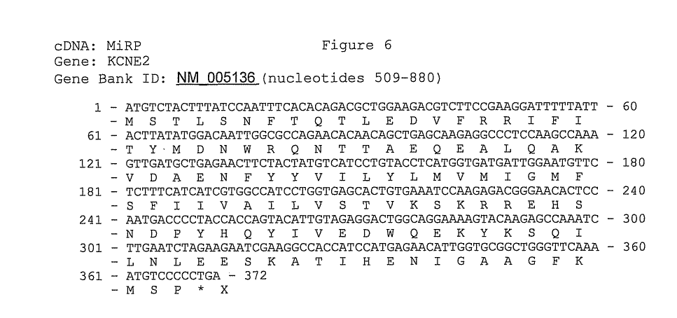

cDNA Sequence List:
SEQ ID NO: 1: KvLQT1 cDNA (GenBank Acc. N^{o} AF000571); SEQ ID NO: 2: KvLQT1 protein (see FIG. 2)
SEQ ID NO: 3: KCNH2 cDNA (GenBank Acc. N^{o} NM000238); SEQ ID NO: 4: KCNH2 protein (see FIG. 3)
SEQ ID NO: 5: SCN5A cDNA (GenBank Acc. N^{o} NM000335); SEQ ID NO: 6: SCN5A protein (see FIG. 4)
SEQ ID NO: 7: KCNE1 cDNA (GenBank Acc. N^{o} NM000219); SEQ ID NO: 8: KCNE1 protein (see FIG. 5)
SEQ ID NO: 9 KCNE2 cDNA (GenBank Acc. N^{o} NM000335); SEQ ID NO: 10: KCNE2 protein (see FIG. 6).

The oligonucleotides of the invention, comprising the mutations identified and characterized in the present invention, are numbered from 11 to 149 and are reported in Table 1.

1-46. (canceled)

47. Method for in vitro diagnosis of the predisposition to the Long QT Syndrome or for the diagnosis of the full-blown Long QT Syndrome, comprising the detection in a DNA sample of a group of non private mutations in KVLQT1, KCNH2 and SCN5A genes, corresponding to the following amino acids or nucleotide positions:
in the KCNQ1 gene, according to the amino acids numbering, the mutations: L137, R174, G179, R190, I204, R231, D242, V254, H258, R259, L262, G269, S277, V280, A300, W305, G314, Y315, T322, G325, A341, P343, A344, R360, R518, R539, I567, R591, R594, and
in the KCNQ1 gene, according to nucleotide numbering, the mutations: 1514+1G>A, (SEQ ID NO: 52), 1513-1514delCA (SEQ ID NO: 53), the mutation 921+1 G>A and the mutation 921+2 T>C;
in the KCNH2 gene, according to amino acid numbering, the mutations: Y43, E58, del82-84IAQ, W412, S428, R534, L552, A561, G572, R582, G604, D609, T613, A614, T623, G628, S660, R752, S818, R823 and
in the KCNH2 gene according to nucleotide numbering, the mutations: 453delC, 453-454insCC, 576delG (SEQ ID NO: 79), 578-582deICCGTG (SEQ ID NO: 80), G2398+3A>G (SEQ ID NO: 110), G2398+3A>T (SEQ ID NO: 111), 3093-3106del (SEQ ID NO: 125), 3093-3099del/insTTCGC identified as (SEQ ID NO: 126), and 3100delC (SEQ ID NO: 128);
in the SCN5A gene, according to the amino acid numbering, the mutations: A413, T1304, P1332, 1505-1507delKPQ, R1623, R1644, Y1767, E1784,
where the presence of at least one change in the sample from wild type correlates with the QT Syndrome or with the predisposition to said syndrome.

48. Method according to claim 47 wherein said mutations are:
in the KCNQ1 gene, according to amino acid numbering: L137F, R174C and R174P, G179S, R190W and R190Q, I204M, R231C and R231H, D242N, V254L and V254M, H258N and H258R, R259C, L262V, G269D and G269S, S277L, V280E, A300T, W305S and W305stop, G314D and G314S, Y315C, T322M, G325R, A341E and A341V, P343C and P343R, A344E, R360T, R518G, R518P, R518stop, R539W, I567T, R591H, R594Q and,
in the KCNQ1 gene, according to the nucleotide numbering, the mutations: 1514+1G>A, (corresponding to the mutation of SEQ ID NO: 52), 1513-1514delCA corresponding to SEQ ID NO: 53, the mutation 921+1 G>A and the mutation 921+2 T>C;
in the KCNH2 gene, according to the amino acid numbering, the mutations: Y43C, E58A and E58G and E58D and E58K, del82-84IAQ, W412stop, S428L, R534C and R534L, L552S, A561T and A561V, G572C and G572D, R582C and R582L, G604S, D609H and D609G, T613M, A614V, T6231, G628S, S660L, R752W, S818L, R823W and,
in the KCNH2 gene, according to the nucleotide numbering, the mutations: 453delC, 453-454insCC, 576delG (SEQ ID NO: 79), 578-582deICCGTG (SEQ ID NO: 80), G2398+3A>G (SEQ ID NO: 110), G2398+3A>T (SEQ ID NO: 111), 3093-3106del (SEQ ID NO: 125), 3093-3099del/insTTCGC (SEQ ID NO: 126), and 3100delC (SEQ ID NO: 128);
in the SCN5A gene, according to the amino acid numbering, the mutations: A413E and A413T, T1304M, P1332L, 1505-1507delKPQ, R1623Q, R1644C preferably SEQ ID NO: 140, Y1767C, E1784K.

49. The method according to claim 48 wherein said mutations are identified with oligonucleotides comprising the following nonanucleotides or complementary sequences thereof:
KCNQ1: SEQ ID NO: 13 (L137F), SEQ ID NO: 16 (R174P), SEQ ID NO: 17 (R190W), SEQ ID NO: 21 (1204M), SEQ ID NO: 24 (R231H), SEQ ID NO: 26 (V254L), SEQ ID NO: 27 (H258N), SEQ ID NO: 28 (H258R), SEQ ID NO: 29 (L262V), SEQ ID NO: 34 (V280E), SEQ ID NO: 39 (T322M), SEQ ID NO: 40 (P343L), SEQ ID NO: 41 (P343R), SEQ ID NO: 46 (R360T), SEQ ID NO: 55 (R518G), SEQ ID NO: 56 (R518P), SEQ ID NO: 59 (1567T), SEQ ID NO: 52, SEQ ID NO: 53;
KCNH2: SEQ ID NO: 67 (Y43C), SEQ ID NO: 69 (E58A), SEQ ID NO: 70 (E58G), SEQ ID NO: 71 (E58D), SEQ ID NO: 75 (del IAQ), SEQ ID NO: 85 (W412stop), SEQ ID NO: 88 (S428L), SEQ ID NO: 97 (G572D), SEQ ID NO: 98 (R852L), SEQ ID NO: 99 (D609H), SEQ ID NO: 106 (S660L), SEQ ID NO: 113 (S818P), SEQ ID NO: 79, SEQ ID NO: 80, SEQ ID NO: 110, SEQ ID NO: 111, SEQ ID NO: 125, SEQ ID NO: 126, SEQ ID NO: 128;
SCN5A: SEQ ID NO: 133 (A413E), SEQ ID NO: 134 (A413T), SEQ ID NO: 140 (R1644C), SEQ ID NO: 142 (Y1767C).

50. The method according to claim 47 wherein said mutations are detected according to one of the following techniques:
restriction pattern of a DNA fragment from the sample comprising the mutation, optionally in parallel with a sample corresponding to the wild type sequence,
hybridization of nucleic acids of the sample with specific probes under selective conditions,
PCR,
Oligonucleotide Ligation Assay,
electrophoresis showing the migration pattern of the nucleic acids of the sample,
direct sequencing,
Denaturing High Performance Liquid chromatography,
wherein, according to each of the above mentioned methods, the presence of a mutation in the sample is optionally further confirmed by comparison with a pattern obtained from control nucleic acids not carrying the mutation (or wild type) or by hybridization under selective conditions.

51. Method according to claim 47 further comprising the sequence characterization of the KVLQT1 (KCNQ1) and/or KCNH2 genes or Open Reading Frames.

52. Method according to claim 51 further comprising the sequence characterization of the SCN5A, KCNE1 and/or KCNE2 genes or Open Reading Frames.

53. Method according to claim 52 wherein the sequence of the Open reading Frames is characterized by direct sequencing with at least one of the oligonucleotide primers listed in Table 4.

54. The method according to claim 47 wherein said sample is genomic DNA.

55. The method according to claim 47 further comprising a step of reverse transcription of a RNA sample into cDNA.

56. Method for prevention of the iatrogenic Long QT Syndrome comprising the identification of mutations in KVLQT1, KCNH2 and SCN5A genes according to claim 47.

57. Method for diagnosis of the iatrogenic Long QT Syndrome comprising the identification of mutations in KVLQT1, KCNH2 and SCN5A genes according to claim 47.

58. Isolated nucleic acid comprising at least one of the oligonucleotides of sequence selected from the group consisting of:
KCNQ1: SEQ ID NO: 13 (L137), SEQ ID NO: 16 (R174P), SEQ ID NO: 17 (R190W), SEQ ID NO: 21 (1204M), SEQ ID NO: 24 (R231H), SEQ ID NO: 26 (V254L), SEQ ID NO: 27 (H258N), SEQ ID NO: 28 (H258R), SEQ ID NO: 29 (L262V), SEQ ID NO: 34 (V280E), SEQ ID NO: 39 (T322M), SEQ ID NO: 40 (P343L), SEQ ID NO: 41 (P343R), SEQ ID NO: 46 (R360T), SEQ ID NO: 55 (R518G), SEQ ID NO: 56 (R518P), SEQ ID NO: 59 (1567T), SEQ ID NO: 52, SEQ ID NO: 53;
KCNH2: SEQ ID NO: 67 (Y43C), SEQ ID NO: 69 (E58A), SEQ ID NO: 70 (E58G), SEQ ID NO: 71 (E58D), SEQ ID NO: 75 (del IAQ), SEQ ID NO: 85 (W412stop), SEQ ID NO: 88 (S428L), SEQ ID NO: 97 (G572D), SEQ ID NO: 98 (R852L), SEQ ID NO: 99 (D609H), SEQ ID NO: 106 (S660L), SEQ ID NO: 113 (S818P), SEQ ID NO: 79, SEQ ID NO: 80, SEQ ID NO: 110, SEQ ID NO: 111, SEQ ID NO: 125, SEQ ID NO: 126, SEQ ID NO: 128;
SCN5A: SEQ ID NO: 133 (A413E), SEQ ID NO: 134 (A413T), SEQ ID NO: 140 (R1644C), SEQ ID NO: 142 (Y1767C).

59. Isolated nucleic acid according to claim 58 having a length comprised between 15 and 30 nucleotides.

60. Isolated nucleic acid having a sequence complementary to the nucleic acids according to claim 58.

61. Two-dimensional or three-dimensional support comprising at least one of the nucleic acids or oligonucleotides according to claim 58.

62. Two-dimensional or three-dimensional support comprising at least one of the nucleic acids or oligonucleotides according to claim 60.

63. A Kit for the detection of mutations in KCNQ1, KCNH2 SCN5A genes comprising at least one of the nucleic acids according to claim 58 or complementary sequence thereof.

64. Kit according to claim 63 comprising oligonucleotides suitable for detection of the following further mutations:
in the KCNQ1 gene, according to the amino acid numbering: R174C, G179S, R190Q, R231C, D242N, V254M, R259C, G269D and G269S, S277L, A300T, W305S and W305stop, G314D and G314S, Y315C, G325R, A341E and A341V, A344E, R518stop, R539W, R591H, R594Q;
in the KCNQ1 gene, according to the nucleotide numbering: mutation 1514+1G>A, mutation 921+1 G>A and mutation 921+2 T>C;
in the KCNH2 gene, according to the amino acids numbering: E58K, W412stop, R534C, L552S, A561T and A561V, G572C, R582C, G604S, D609G, T613M, A614V, T623I, G628S, R752W, S818L, R823W;
in the KCNH2 gene, according to nucleotide numbering: 453delC, 453-454insCC;
in the SCN5A gene according to amino acids numbering: T1304M, P1332L, 1505-1507delKPQ, R1623Q, E1784K.
